(19) United States
(12) Patent Application Publication
(10) Pub. No.: US 2002/0007832 A1
(43) Pub. Date: Jan. 24, 2002

(54) AUTOMATED CARDIOPULMONARY RESUSCITATION INSTRUCTION DEVICE

(76) Inventor: JAMES DOHERTY, GALVA, IL (US)

Correspondence Address: REESE TAYLOR ESQ, RENNER KENNER GRIEVE BOBAK TAYLOR, & WEBER, SIXTEENTH FLOOR FIRST NATIONAL TOWER, AKRON, OH, 443081456

(21) Appl. No.: 09/422,218

(22) Filed: Oct. 21, 1999

(51) Int. Cl. A62B 7/00; A62B 9/00; A61M 15/00

(52) U.S. Cl. 128/202.29; 128/200.24; 128/203.11

(57) ABSTRACT

A device for assisting in the administration of cardiopulmonary resuscitation includes a case having a latch that is normally closed. The case carries a circuit which has a switch that is normally open. The switch is coupled to the latch and is closed when the case is opened. The case includes a rescue aid, such as a ventilation mask, wherein opening of said latch causes closure of said switch which energizes an instruction circuit that immediately provides verbal instructions for performing cardiopulmonary resuscitation and use of the rescue aid.

### RELATED PATENT APPLICATIONS

None.

### FIELD OF THE INVENTION

This invention relates in general to safety devices and, in particular, to devices which provide cardiopulmonary resuscitation instructions to a lay-person rescuer.

### BACKGROUND OF THE INVENTION

Cardiopulmonary rescue procedures have been developed by the American Heart Association in conjunction with the American Red Cross. As is well known, cardiopulmonary resuscitation (CPR) is a combination of artificial respiration and artificial circulation utilized as an emergency procedure when cardiac arrest occurs. When properly performed, CPR can save lives.

Proper administration of CPR requires the rescuer to perform a series of physical procedures that cause artificial breathing and circulation. These procedures, such as chest compressions, are called for in prescribed sequences and with specific timing. The sequence and timing are important because they are directly related to a standard pulse beat needed to prevent death.

A device for assisting and administering CPR is disclosed in U.S. Pat. RE. 34,800, which is incorporated herein by reference. This patent teaches a prompting device for those previously trained in CPR with annunciated and sequenced instructions on the steps that need to be taken to properly administer CPR. The primary benefit of such a system is that it allows for input of various scenarios as to the age and status of the patient. Such a device also reminds the rescuer of very important steps which are required to effectively administer CPR. This need is evident for those individuals who have received CPR training, but have not recently applied their knowledge. As such, the prompting device supplements the rescuer's memory and assists in their effective administration of CPR in a stress-felt situation.

Although the device disclosed in the RE. 34,800 patent is presumably effective for its stated purpose, the device has several deficiencies. Primarily, for untrained personnel, operation of the device is complex and requires the user to input various information prior to the actual annunciation of the prompting steps to assist the person in need. In particular, the device includes at least 10 input buttons directed to whether the patient is an infant, child or adult, and as to whether or not the patient is choking or not. In addition, for those people who may be blind, vision impaired, or farsighted, which accounts for some people or spouses of those who may need cardiopulmonary resuscitation, it is believed that the device disclosed in RE 34,800 is constructed in such a way as to be a detriment for use by the visually impaired population. It is submitted that in an emergency situation which involves highly stressful emotions, that inputting information into the 10 buttons is a serious detriment to fast and efficient treatment. It is submitted that in times of stress, it is better to provide a simple device with only one scenario which addresses the most common situation, but also follows the intent of the Americans with Disability Act. Sudden Cardiac Arrest (SCA) accounts for 400,000 to 500,000 deaths per year in the United States, and typically afflicts an adult person who suddenly loses the ability to breathe and whose heart suddenly and virtually simultaneously stops beating.

Therefore, it has become apparent that it is desirable to have a simple, automated cardiopulmonary resuscitation instruction device which provides verbal, or if desired, visual instructions on administering CPR. It is also desirable that this device provide additional components or aids for assisting in the administration of CPR.

### SUMMARY OF THE INVENTION

It has been found, therefore, that an automated cardiopulmonary resuscitation instruction device can be provided which generates verbal or visual instructions for use in administering CPR. In particular, the device provides a latched case which, when opened, initiates the dissemination of instructions. Moreover, the device includes other items which facilitate the safe and effective administration of CPR.

Specifically, it has been found that a case, which has a simple latching mechanism, includes a normally open switch coupled to the latching mechanism when the case is closed. When the case is opened by a rescuer to administer CPR, the switch is closed and verbal or visual instructions are provided. In particular, the switch is coupled to an instruction circuit energized by closure of the switch. The circuit includes a power supply and a processor with a voice memory chip that begins disseminating instructions upon opening of the case.

It has also been found that the instruction circuit includes a speaker for annunciating the instructions carried on the voice chip. Or, in the alternative, the instruction circuit may illuminate a liquid crystal display with written or pictorial instructions upon opening of the case.

It has also been found that the case can carry other items such as a ventilation mask for mouth-to-mouth resuscitation, latex-free gloves so as to preclude spreading of infectious diseases, and anti-microbial wipes to further reduce the spread of disease.

It has also been found that the device can effectively direct a potential layperson rescuer in one-rescuer CPR. In particular, the device is simple to use and does not need input from the lay-rescuer. By virtue of using a basic well-known heart-shape case, the system automatically activates when the rescuer accesses any of the barrier protections carried by the case. By doing this, there is no conscious input, no menu to follow, and no selections to be made. Upon opening the case to access one of the aids, the voice chip instructs the rescuer in one person CPR using an accepted American Heart Association protocol. It is envisioned that the voice chip could also provide instructions for administering child or infant CPR. This can be accomplished by providing a single device for each CPR scenario. Alternatively, a switch could be provided to allow selection of one of the three CPR scenarios. Ideally, this switch would be set at the factory during manufacture of the device.

Accordingly, production of an automated cardiopulmonary resuscitation device of the character above described becomes the principal object of this invention with other objects thereof, becoming apparent upon a reading of the following brief specification considered and interpreted in view of the accompanying drawings.

### BRIEF DESCRIPTION OF THE DRAWINGS

FIG. 1 is a schematic view of the automated cardiopulmonary resuscitation instruction device and, in particular, a case shown in an open position;

FIG. 2 is a schematic electrical circuit diagram of the instruction circuit employed in the instruction device; and

FIG. 3 is an operational flow chart showing operation of the instruction device.

### BRIEF DESCRIPTION OF THE PREFERRED EMBODIMENTS

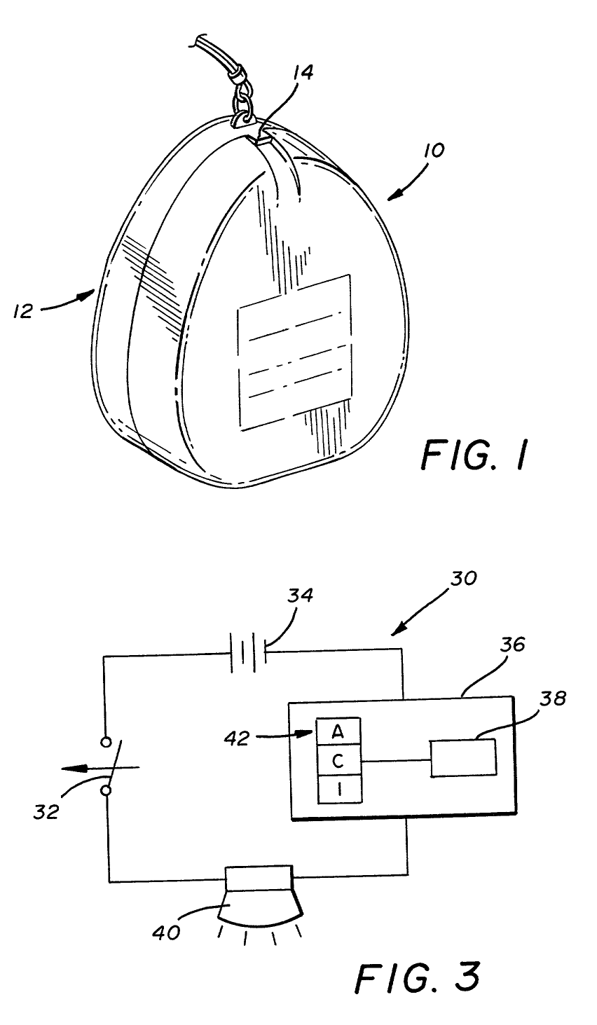

Referring first then to FIG. 1 of the drawings, it will be seen that an automated cardiopulmonary resuscitation (CPR) instruction device, generally indicated by the numeral 10, includes a case 12, preferably made of thermoplastic or similar material. The case 12 provides a hinge 13 interconnecting the two halves of the case such that each is pivotable with respect to the other. At an end opposite the hinge 13, the case 12 provides a latch 14 such that opening and closing of the case requires deflectable movement of the latch 14. When closed, the case essentially seals all contents contained therein and protects the integrity of each component.

The case 12 may carry a pair of latex-free gloves 16, packet(s) of anti-microbial wipes 18, and a ventilation mask 22 which is used in the administration of mouth-to-mouth breathing when needed. A reminder card 24 may also be included with instructions on the administration of CPR and the like. It may also contain a written reminder to the rescuer to call emergency personnel.

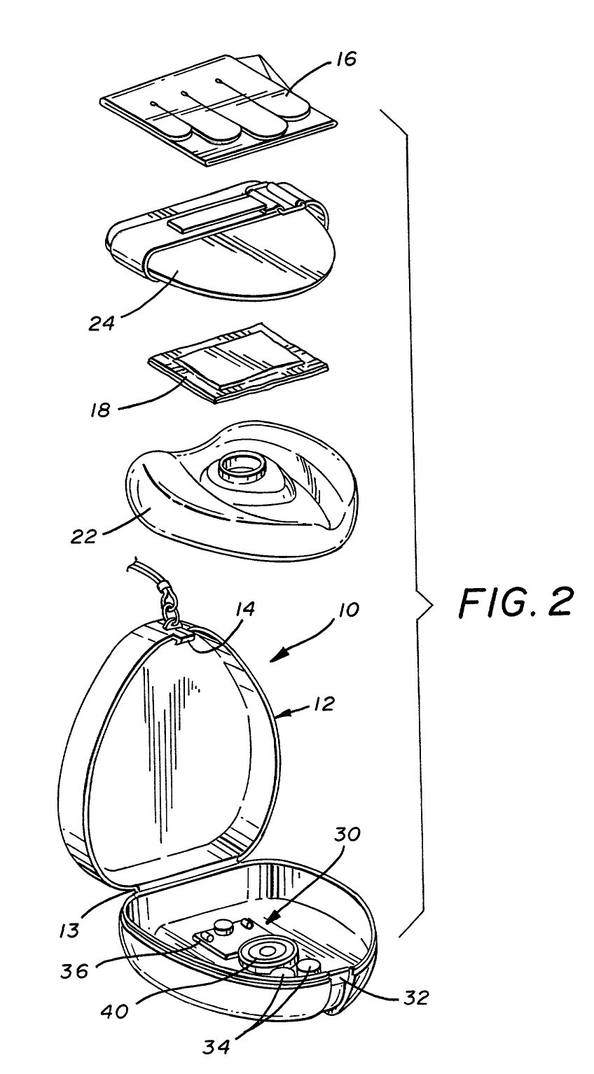

Referring now to FIGS. 1 and 2, it can be seen that an instruction circuit, designated generally by the numeral 30, is carried in one of the halves of the case 12. Typically, the instruction circuit 30 is implemented on a printed circuit board and mounted to the case in a manner well-known in the art. Also carried by the case 12 is a normally-open switch 32 which is coupled to the latch 14. In other words, when the case 12 is closed and the latch 14 is engaged, the switch 32 is in an open position. As soon as the latch 14 is opened so as to separate the halves of the case 12, the switch 32 is closed. Upon closure of the switch 32, the instruction circuit 30 is energized by a power supply 34. The power supply 34 is typically a commercially available battery with a long shelf life.

A processor 36 is included in the instruction circuit 30 and incorporates a voice chip 38. The processor 36 is connected to a voice speaker 40 such that upon closure of the switch 32, the information contained in the voice chip 38 is annunciated over the speaker 40 to the rescuer. The processor 36 contains the necessary hardware, software, and memory to ensure operation of the device 10. Although not shown in the drawings, it will be appreciated by those skilled in the art that written or pictorial instructions could be provided to the user on a liquid crystal display or the like, in the same sequence as the verbal instructions. In the preferred embodiment, use of verbal instructions allows for the rescuer to devote full visual attention to the patient.

A three-scenario selector switch 42 may be connected to the voice chip 38. Ideally, the selector switch 42 is set at the factory and the case 12 is provided with appropriate indicia indicative of the scenario selected. Alternatively, the switch 42 could be set by the user prior to use in the field. The selector switch 42 provides for selection of three different scenarios, wherein the “A” designation is for instructing the user in administering CPR to an adult. Likewise, the “C” and “I” designations toggle the voice chip 38 to provide the CPR instructions for a child or an infant, respectively.

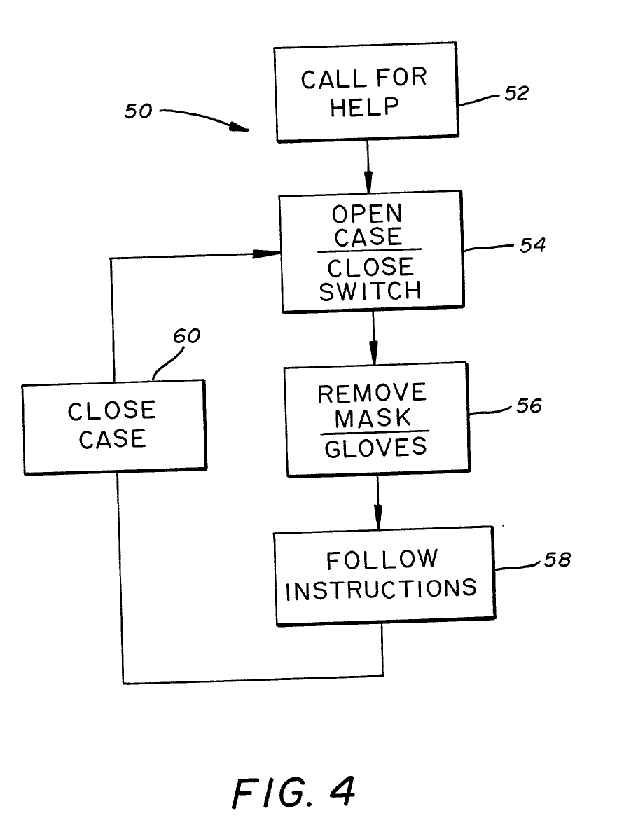

Referring now to FIG. 3, it can be seen that an operational flow chart for use of the device 10 is presented, wherein the operational steps are generally designated by the numeral 50. At a first step 52, the individual should call for help. Next, at step 54, the user opens the case which automatically closes the switch 32 and initiates annunciation of the verbal instructions for administering CPR. At step 56, the rescuer is instructed to remove the ventilation mask 22 and to wear the gloves 16 that are included in the case 12. Following this, at step 58, the user follows the instructions annunciated by the instruction circuit 30. Alternatively, someone could read the instructions from the reminder card. These instructions assist the lay-person rescuer in administering CPR for the most common type of cardiac arrest. Of course, other situation-specific instructions could be provided on the voice chip 38. In the event that certain instructions are missed, the user may close the case at step 60 which returns the operation of the instruction circuit 30 to the beginning, whereupon the user could re-open the case and start anew.

It is apparent then from the above description of the structure and operation of the device 10 that the problems associated with previous CPR assisting device have been overcome. In particular, the device 10 provides a simple mechanism for annunciating CPR instructions upon opening of a case. This procedure is advantageous in that the use of the device is in a normally stressfully situation and distractions associated with inputting various scenarios is eliminated. Moreover, the device is advantageous in that it contains other elements required for appropriate administration of CPR.

While a full and complete description of the invention has been set forth in accordance with the dictates of the Patent Statutes, it should be understood that modifications can be resorted to without departing from the spirit hereof or the scope of the appended claims. For example, the invention has been described in the context of a cardiopulmonary resuscitation instruction device. However, it is believed apparent that the operational circuitry could be readily adapted to provide instructions for other emergency-type situations in which the full attention of the rescuer can be directed to the patient while the instructions are annunciated.

What is claimed is:

1. Apparatus for providing cardiopulmonary resuscitation instructions, comprising
a) a case;
b) an instruction circuit carried by said case;
c) a switch carried by said case and coupled to said instruction circuit;
d) wherein actuation of said switch energizes said instruction circuit without further user input and which in turn provides instructions to assist in performing cardiopulmonary resuscitation.

2. The apparatus of claim 1, wherein said case includes a latch that is coupled to said switch, wherein opening of said latch closes said switch to energize said instruction circuit and annunciates the instructions.

3. The apparatus of claim 2, wherein said instruction circuit includes a voice circuit connected to a speaker which annunciates the instructions provided.

4. The apparatus of claim 3, wherein said case carries a ventilation mask and a pair of gloves.

5. The apparatus of claim 1, wherein said instruction circuit includes a multi-scenario selector switch which pre-selects the instructions to be provided to the end-user.

6. A kit for assisting in the administration of cardiopulmonary resuscitation, the kit comprising:
a) a case having a latch, wherein said case is normally closed;
b) a circuit carried in said case, said circuit having a switch that is normally open, said switch coupled to said latch; and
c) a rescue aid carried in said case, wherein opening of said latch causes closure of said switch which energizes said circuit, said circuit immediately providing verbal instructions for use of said rescue aid and in performing cardiopulmonary resuscitation.

7. The kit according to claim 6, wherein said case also carries gloves to be worn by the user of the kit.

8. The kit according to claim 7, wherein said case also carries an anti-microbial wipe for use by the user.

9. The kit according to claim 6, wherein closure and then re-opening of the case re-starts the verbal instructions.

10. The kit according to claim 6, wherein said circuit provides a scenario selector switch for selection of patient-specific instructions.
